(19) United States
(12) Patent Application Publication
(10) Pub. No.: US 2022/0104887 A1
(43) Pub. Date: Apr. 7, 2022

(54) SURGICAL RECORD CREATION USING COMPUTER RECOGNITION OF SURGICAL EVENTS

(71) Applicant: Asensus Surgical US, Inc., Durham, NC (US)

(72) Inventors: Kevin Andrew Hufford, Durham, NC (US); Caleb T. Osborne, Durham, NC (US); Lior Alpert, Haifa (IL)

(21) Appl. No.: 17/495,792

(22) Filed: Oct. 6, 2021

Related U.S. Application Data
(60) Provisional application No. 63/088,393, filed on Oct. 6, 2020.

(51) Int. Cl. A61B 34/20; A61B 34/30; A61B 34/00; A61B 90/00

(52) U.S. Cl. CPC A61B 34/20; A61B 34/30; A61B 2034/256; A61B 90/37; A61B 2034/252; A61B 34/25

(57) ABSTRACT

A system for capturing records of a surgical procedure includes a camera that captures real time video images of a surgical treatment site. The system detects completion of a predetermined step of a surgical procedure by analyzing the image data using computer vision or analyzing kinematic information from a robotic component. Upon detecting completion of the predetermined step, the system stores a frame from the image data in a surgical record for the procedure.

### BACKGROUND

This application relates to the use of computer vision to recognize events or procedural steps within a surgical site. Surgical systems providing this type of feature can reduce the surgeon's cognitive load by recognizing and alerting the surgeon to the detected events within the surgical site, by performing tasks the surgeon would otherwise need to perform (such as creating documentation recording the detected events, or adjusting system parameters), by adjusting aspects of the visual augmentation of the surgical site display, and/or by providing contextually-aware information to the surgeon when appropriate, and (in the case of on-screen display) causing that information to disappear when it is no longer needed. Reducing the surgeon's cognitive load can shorten the surgical procedure, thereby reducing risk of complications, lowering procedure costs, and increasing surgeon productivity.

Co-pending and commonly owned U.S. Ser. No. 17/368,753, filed Jul. 6, 2021, and entitled “Providing Surgical Assistance via Automatic Tracking and Visual Feedback During Suture” describes examples of ways in which computer vision is used to give feedback and assistance to a user performing suturing within the field of vision of a camera capturing images of a surgical site.

This application describes a system that creates surgical records in response to computer recognition of steps or events of a surgical procedure based on computer vision analysis or kinematic data.

### BRIEF DESCRIPTION OF THE DRAWINGS

FIG. 1 is a block diagram schematically illustrating a system according to the disclosed embodiments.

FIG. 2 is a sequence of steps schematically illustrating an embodiment of a method using computer vision to recognize and document procedural events;

FIGS. 3A-3D show a sequence of screen captures of an image display during use of the first embodiment;

FIG. 4 is a flow diagram illustrating an example of a procedural event detection and response method.

### DETAILED DESCRIPTION

System

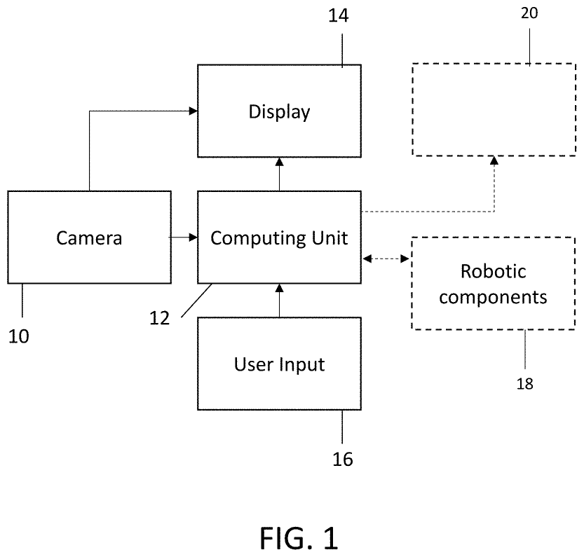

A system useful for performing the disclosed methods, as depicted in FIG. 1, may comprise a camera 10, a computing unit 12, a display 14, and, preferably, one or more user input devices 16. The system is optionally used with a robot-assisted surgical system using one or more robotic components 18, such as robotic manipulators that move the instruments and/or camera, and/or robotic actuators that articulate joints, or cause bending, of the instrument or camera shaft.

The camera 10 is one suitable for capturing images of the surgical site within a body cavity. It may be a 3D or 2D endoscopic or laparoscopic camera. Where it is desirable to use image data to detect movement or positioning of instruments, or tissue in three dimensions, configurations allowing 3D data to be captured or derived are used. For example, a stereo/3D camera might be used, or a 2D camera with software and/or hardware configured to permit depth information to be determined or derived using structured light techniques. The terms “camera,” “endoscopic camera,” and “laparoscopic camera” may be used interchangeably in this description without limiting the scope of the invention.

The computing unit 12 is configured to receive the images/video from the camera and input from the user input device(s). If the system is to be used in conjunction with a robot-assisted surgical system in which surgical instruments are maneuvered within the surgical space using one or more robotic components the system may optionally be configured so that the computing unit also receives kinematic information from such robotic components 18 for use in recognizing procedural steps or events as described in this application.

An algorithm stored in memory accessible by the computing unit is executable to, depending on the particular application, use the image data to perform one or more of the functions described with respect to the first and second embodiments.

The system may include one or more user input devices 16. When included, a variety of different types of user input devices may be used alone or in combination. Examples include, but are not limited to, eye tracking devices, head tracking devices, touch screen displays, mouse-type devices, voice input devices, foot pedals, or switches. Eye-tracking, head-tracking, mouse devices or other devices that cause movement of a cursor on a display may be used by a user to move a cursor around the display to identify regions of interest, to position the cursor on icons displayed and representing options the user can select. Various movements of an input handle used to direct movement of a robotic component of a surgical robotic system may be received as input (e.g., handle manipulation, joystick, finger wheel or knob, touch surface, button press). Another form of input may include manual or robotic manipulation of a surgical instrument having a tip or other part that is tracked using image processing methods when the system is in an input-delivering mode, so that it may function as a mouse, pointer and/or stylus when moved in the imaging field, etc. Input devices of the types listed are often used in combination with a second, confirmatory, form of input device allowing the user to enter or confirm (e.g., a switch, voice input device, foot pedal, button, icon to press on a touch screen, etc., as non-limiting examples).

Some hospitals or surgical centers have operating room dashboards or notification systems 20 that provide updates to hospital personnel or waiting room occupants as to the expected remaining duration of the procedure. The one or more computing units may further be configured to interact with such a system to keep the relevant parties apprised of the expected time remaining for the surgical procedure.

### First Embodiment

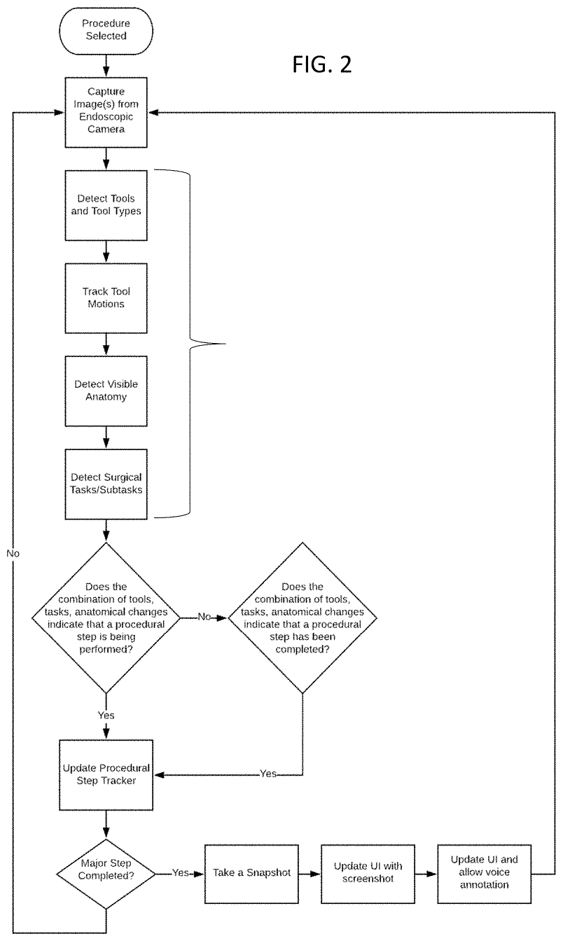

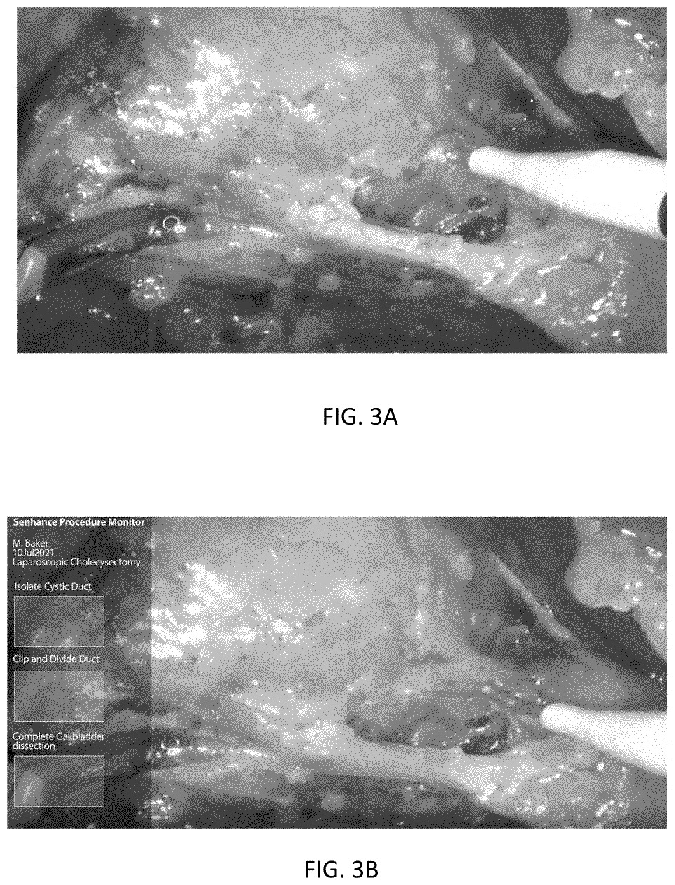

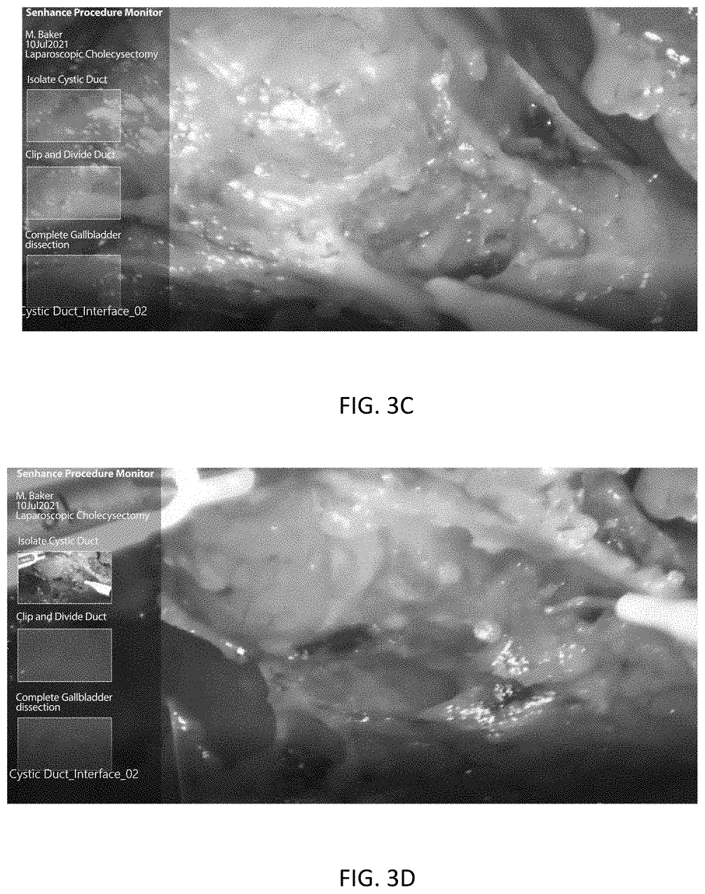

FIG. 2 illustrates a method that may be performed by a first embodiment of the described system. While not required, in some cases, the system is configured to initially receive user input given to instruct the system to enter into a mode of operation within which tasks, steps etc. of a particular predetermined procedure are being monitored for. For example, in the specific example described with respect to FIGS. 3A-3D, user input may be given to instruct the system to enter a mode of operation by which steps of a cholecystectomy are performed.

The initial step or set of steps relate to receiving data from which the computing unit can determine the occurrence of a surgical tasks, subtasks, or events. Images are captured from the camera 10 as shown. The system may optionally also receive kinematic data for any robotic components 18 being used to perform the surgery, such as data based on input generated by sensors of the robotic components.

The system executes algorithms that analyze the image data and other data to carry out any one or combination of the following:
Detecting surgical tools within the surgical site using computer vision.
Identifying the tool types of the tools within the surgical site. This may be performed using computer vision and the image data, but it can also be performed using other features of the system alone or in combination with the computer vision. For example, with some robotic systems, instruments mounted to manipulators include RFID tags, EEPROMS, or other electronic, electrical, optical, mechanical etc. identifiers that are read or detected by the robotic system to identify the tool type to the system. In these types of embodiments, the system might identify tools using the image data, then if more than one type of tool is in the surgical site, prompt the user to confirm which instrument is which instrument type. For example, if the system knows based on the above methods that instrument type A and instrument type B are in use, a visual prompt (e.g. a text or image overlay on the display) or an auditory prompt might solicit input from the user confirming which of the instruments is the type A or B instrument. As a particular example, the user might be prompted to press a button on the user input device assigned to control the type A instrument, or to touch the type A instrument on a touch screen display. In other systems (whether robotic or not), the tools are detected in the surgical site using computer vision, and the user is prompted to give input (e.g. selecting from a memo, saying the name of the tool type so it is input to the system using a microphone) identifying the tool type for each one.
Tracking tool motions within the surgical site. This may be done using computer vision to track the motions of the instruments using the image data, and/or it may use kinematic data from robotic components.
Detecting anatomy or anatomical features within the surgical site. This step, performed using computer vision, may include detecting predetermined organs, vessels, or parts of organs. It may also include detecting changes to certain anatomy during the course of a procedure, such as changes that would indicate a step has been carried out. For example, in the example discussed below, the cystic duct may be identified. Isolation or mobilization of the cystic duct may also be a detected change to that anatomy. Tissue damage using cutting tools or energy (e.g. excision, ablation etc.) may be another change to anatomy that is detected using color changes and/or changes in shape. See, for example, U.S. application Ser. No. 17/368,756, filed Jul. 6, 2021, entitled “Automatic Tracking of Target Treatment Sites Within Patient Anatomy”. In some cases, both the type of anatomy is detected and the change are identified. In others, only the changes are detected. Evidence of changes could also be detected using computer vision, such as the presence of smoke.

The system then recognizes/detects whether tool movement patterns, sequences of movements, detected changes etc. indicate that a predetermined surgical task or subtask is being, or has been, performed. For example, recognizing the motion of a tool being used to wrap a suture around another tool allows the system to detect that the surgeon is in the process of tying a suture knot. Where a needle-holder is known or recognized as being controlled by a user's right hand, and a grasper is known or recognized as being controlled by the left hand but is then detected as being replaced with scissors, the system might detect that the surgeon has just completed the process of tying a knot. A database associated with the computing unit may store sequences of tasks and/or sub-tasks for a given procedure. Some may be steps of standard procedures with accepted best practices. Others may be tasks/sub-tasks of a custom procedure developed by a surgeon or team and input or saved to the system by a user. Such customized procedures might be patient-specific procedure steps developed based on the co-morbidities, prior surgeries, complications, etc. of the patient who is to be treated.

A database associated with the one or more computing units preferably includes a tracker that is updated as each task/sub-task is detected during a surgical procedure. If the system determines that the detected tool movement patterns, sequences of movements, detected changes etc. indicate that a predetermined surgical task or subtask is being, or has been, performed, the tracker is updated. Examples of tasks that might be tracked include access to or exposure of a treatment site, dissection, and suturing. More significant steps or procedures that might be part of, or steps marking the completion of, the procedural plan might include tissue or organ removal steps (i.e. “-ectomy” procedures), tissue cutting or formation of openings (“-otomy” procedures) etc.

The procedures stored in the database might have designated “major” steps or tasks, which for the first embodiment are defined as steps identified to the system as ones for which photo documentation is to be captured upon their completion. If the detected surgical task or sub-task is not a “major” task, the process repeats for the next task of the procedure or planned sequence of tasks. If it is a “major” task, a snapshot (e.g. a frame grab from the video image) is captured. The capture may be automatically performed by the system, or the user may take an action (optionally in response to a prompt to do so) that inputs instructions to capture it (e.g. by giving a voice command, touching a graphical user interface or, if the surgeon is at a robotic surgery console, by a button press). The snapshot is displayed on the screen as an overlay of the displayed endoscopic image and/or stored in the database for use in creating records of the surgery. By storing the snapshot, it is meant that a still image is saved independently of any video of the procedure that may be stored. As one example discussed in connection with FIGS. 3A-3D, the user interface may be configured with overlays to list major tasks, and to overlay the captured snapshots associated with the listed tasks on the image display. This will be discussed in connection with FIGS. 3A-3D. Once the snapshot of the major task completion is captured, the process repeats until all tasks of the procedure have been completed.

In addition to capturing the snapshot, the system may prompt the user to record a verbal annotation in real time. The verbal annotation is stored in a database and is associated with the snapshot, so the two may become part of a record of the procedure and/or subsequently used to train a machine learning algorithm.

The system may additionally or alternatively be configured to perform other functions in response to the scene detected. For example, where robotic components are used, it may be beneficial to set different force thresholds or scaling factors (between the user input and the instrument motion) depending on the steps being performed or the instruments being used. As one example, if the system detects that a 3 mm needle holder has been introduced, but it does not detect the presence of a suture needle in the scene captured in the images, the maximum force threshold for robotic control of that instrument may be set to a relatively lower limit. On the other hand, if the system detects that a suture needle is introduced into the scene, the maximum force threshold is raised to allow so that the needle holder will be able to hold the needle with ample holding force.

In many cases the above-listed steps may utilize a machine learning algorithm such as, for example, one utilizing neural networks that analyzes the images. Such algorithms can be used to recognize tools, and/or anatomic features, and/or recognize when movement patterns, sequences of movements, etc. are those of a predetermined surgical task or subtask. Certain surgical procedure steps require particular motion patterns or sequence of motions. The machine learning algorithm may thus be one that recognizes such patterns of motion and identifies them as being those for a particular surgical task. Movements taken into consideration may be movements of a tool alone or relative to a second tool, or movements of a tool relative to an identified feature of the anatomy. Others might include the combined movement patterns of, or interactions between, more than one tool. In some cases, multiple movements, or steps in a sequence of movements, are recognized as a predetermined surgical task or subtask if performed within a given length of time. Tool exchanges may also be recognized as being part of a recognized surgical task or subtask. As discussed above, detection of tool types that would be used to recognize tool exchanges may use computer vision or information from robotic components, as described above.

### Example

An example of use of the first embodiment will next be described with respect to FIGS. 3A-3D, which show the endoscopic image captured by the camera, as it might be displayed on the display, during a cholecystectomy procedure. In FIG. 3A, the two instruments shown at the surgical site are being used to isolate the cystic duct. During the procedure, a list of procedural tasks may be displayed as an overlay on the screen. In this example, the major tasks of “isolate the cystic duct”, “clip and divide duct,” and “complete gallbladder dissection” are shown. In other examples, both major tasks (which will trigger capture of a snapshot) and other tasks (which will not) may be displayed. The list may disappear and reappear at times to avoid obscuring the surgeon's view of the surgical site. In some embodiments, the system causes the list to appear only when useful to the surgeon, such as during the transitions from completion of one task to initiation of the next task.

The system, using computer vision, recognizes the cystic duct, and displays an overlay over it, as shown in FIG. 3B. The color of that overlay may change as the computer vision algorithm detects changes at the surgical site. For example, the overlay may be a first color (here shown in green; FIG. 3B) when the instruments are spaced from the cystic duct, and a second color (in this case orange; FIG. 3C) when an instrument is detected to be in contact with the cystic duct. A number of different factors may be used to recognize that the instrument is in contact with the cystic duct. It may be based on the determined positions of the duct and the instrument, on movement of the duct, and/or movement of the duct in response to movement of the instrument towards it. Force feedback from a robotic component might also be used in combination with other sources of data such as computer vision.

FIG. 3D shows the display just after the cystic duct has been isolated. A snapshot from the video images has been captured and displayed as an overlay adjacent to the “isolate cystic duct” task. Referring back to FIG. 2, this marks completion of a “major task.” The snapshot may also be saved to the database for use in creating a record of the procedure. The surgeon may be prompted at this point to verbally dictate a notation to be stored in conjunction with the snapshot.

As discussed in connection with FIG. 2, non-major tasks may also be tracked during the course of the procedure, with the procedural step tracker being updated each time a procedural step has been recognized as completed.

In a variation of the FIG. 2 embodiment, input may be given by the user to cause the procedural step tracker to be updated. For example, non-major and major procedural steps may be listed on the overlay, and the user may give touch screen input, verbal input, or any other form of input to “check off” a listed step.

### Second Embodiment

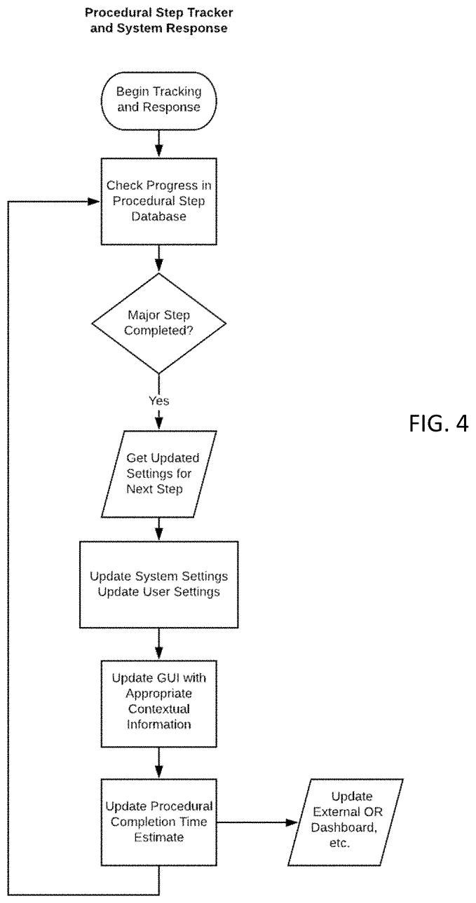

FIG. 4 is a flow diagram giving an example of a procedural step tracking and response process. The process shown in FIG. 4 may be used in conjunction with the process shown in FIG. 2, or it may exclude certain steps (e.g. snapshot capture or display).

In this embodiment, the procedures stored in the database have estimated completion times for certain steps (labeled “major steps” in FIG. 4) and for the overall procedure. If it is determined that a major step has been completed (completion having been recognized by the system as described above and/or confirmed by the user), system settings may be updated for the next major step. As described above, where robotic components are to be used, such settings may be force thresholds or scaling factors for the robotic components. In other cases (whether using robotic components or not), such settings might be endoscope settings, such as zoom level, color, or brightness adjustment, etc. The display is updated so that contextual information relating to the next procedural step is displayed. The estimated procedure completion time is adjusted in the database based on whether the previously completed step was completed in more or less than its estimated completion time. The operating room dashboard or notification system 20 (FIG. 1) is updated to reflect the currently estimated completion time.

The concepts described in this application aid allow the surgeon's cognitive load to be reduced in in a variety of ways. These include providing auto-documentation capability, automatically adjusting system settings, and providing contextually-aware information (such as procedural task lists relevant to the procedure currently underway) to the surgeon.

All patents and applications described herein, including for purposes of priority, are incorporated by reference.

What is claimed is:

1. A system for capturing records of a surgical procedure, comprising:
a camera positionable to capture real time image data of a surgical treatment site;
at least one processor and at least one memory, the at least one memory storing instructions executable by said at least one processor to:
detecting completion of a predetermined step of a surgical procedure by performing at least one of (i) analyzing the image data using computer vision; and (ii) analyzing kinematic information from a robotic component,
upon detecting completion of the predetermined step, storing a snapshot from the image data in the at least one memory.

2. The system of claim 1, wherein the instructions are further executable by said at least one processor to, upon detecting completion of the predetermined step, prompting a user to dictate a note, recording a dictated note, and storing said dictated note.

3. The system of claim 1, wherein the system further includes an image display displaying real time images from the camera, and wherein said instructions are further executable by said at least one processor to display the snapshot as an overlay on the display of the real time image.

4. A system for assisting a surgical practitioner, comprising:
a camera positionable to capture real time image data of a surgical treatment site;
an image display displaying real time images from the camera
at least one processor and at least one memory, the at least one memory storing instructions executable by said at least one processor to:
display, as an overlay on the display, procedural step information;
detect completion of a predetermined step of a surgical procedure by performing at least one of (i) analyzing the image data using computer vision; and (ii) analyzing kinematic information from a robotic component,
upon detecting completion of the predetermined step, update the displayed procedural step information based on completion of the step.

5. A system for assisting a surgical practitioner, comprising:
a camera positionable to capture real time image data of a surgical treatment site;
at least one processor and at least one memory, the at least one memory storing instructions executable by said at least one processor to:
detect completion of a predetermined step of a surgical procedure by performing at least one of (i) analyzing the image data using computer vision; and (ii) analyzing kinematic information from a robotic component,
upon detecting completion of the predetermined step, updating settings of at least one of the camera or a robotic component in preparation for performance of a step subsequent to the detected step.
